(19) United States
(12) Patent Application Publication
(10) Pub. No.: US 2026/0265683 A1
(43) Pub. Date: Sep. 10, 2026

(54) BACILLUS LICHENIFORMIS, USE THEREOF IN DEGRADING POLYOLEFIN PRODUCTS, POLYOLEFIN DEGRADATION MICROBIAL AGENT, PREPARATION METHOD FOR POLYOLEFIN PRODUCT ADDITIVE, AND POLYOLEFIN PRODUCT

(71) Applicants: CHINA INTERNATIONAL MARINE CONTAINERS (GROUP) CO., LTD., Shenzhen, Guangdong (CN); CIMC CONTAINERS (GROUP) CO., LTD., Dongguan, Guangdong (CN); SHENZHEN CIMC ECO MATERIAL DEVELOPMENT CO., LTD., Shenzhen, Guangdong (CN)

(72) Inventors: Song LIU, Shenzhen, Guangdong (CN); Haibo DENG, Shenzhen, Guangdong (CN); Zhilin ZHOU, Shenzhen, Guangdong (CN); Xiaofeng LI, Shenzhen, Guangdong (CN)

(21) Appl. No.: 19/165,363

(22) PCT Filed: Mar. 15, 2024
(86) PCT No.: PCT/CN2024/082040

§ 371 Date: Sep. 15, 2025

(30) Foreign Application Priority Data
Mar. 15, 2023 (CN) 202310246968.1

(51) Int. Cl. C12N 1/205; B29B 17/04; C12F 3/02; C12N 1/04; C12N 11/14; C12R 1/10

(52) U.S. Cl. CPC C12N 1/205; B29B 17/04; C12F 3/02; C12N 1/04; C12N 11/14; B29B 2017/0424; B29K 2023/06; C12R 2001/10

(57) ABSTRACT

Provided is Bacillus licheniformis M0, preserved in Guangdong Microbial Culture Collection Center on 8 Feb. 2023, with the accession number being GDMCC NO: 63160. The present invention further relates to the use of Bacillus licheniformis M0 in degrading polyolefin products, a polyolefin degradation microbial agent, a method for preparing a polyolefin product additive and a polyolefin product.

### BACKGROUND

The present application relates to the technical field of microbial technology, and in particular to Bacillus licheniformis and its use in the degradation of polyolefins.

Common plastics are characterized by structural stability and excellent physical properties, but most of the existing degradation technologies are unable to degrade them, thus causing white pollution after use and disposal. The common “degradable” plastics have the characteristics of not being resistant to high temperatures and being fragile and prone to cracking, and are expensive, making it difficult to use them on a large scale; in particular, the traditional degradable plastics used for films and disposable plastic products have poor cost-effectiveness due to their inferior physical properties compared to polyolefins.

Therefore, there is a need to develop degradable polyolefin products with higher physical properties to at least partially solve the above-mentioned problems.

### SUMMARY

A series of simplified concepts is introduced into the portion of the Summary, which would be further illustrated in the portion of the detailed description. The Summary of the present application does not mean attempting to define the key feature and essential technical feature of the claimed technical solution, let alone determining the protection scope thereof.

To at least partially solve the above-mentioned problem, the present application provides Bacillus licheniformis (Bacillus licheniformis) M0, deposited in the Guangdong Microbial Culture Collection Center on Feb. 8, 2023, with the accession number being GDMCC NO: 63160.

Further, there is provided a use of Bacillus licheniformis M0 in degrading polyolefin products.

Preferably, Bacillus licheniformis M0 is added in the polyolefin product in an amount of more than or equal to 10 cfu/g relative to the total weight of the polyolefin product.

Preferably, Bacillus licheniformis M0 is added in the polyolefin product in an amount of 10³ to 5×10⁴ cfu/g relative to the total weight of the polyolefin product.

Further, there is provided a polyolefin degradation microbial agent, comprising Bacillus licheniformis M0.

Preferably, the polyolefin degradation microbial agent is a polyolefin product additive in the form of a masterbatch.

Preferably, the polyolefin product additive in the form of the masterbatch comprises 50 to 80% of a carrier, 16 to 46% of a polyolefin resin, and 4% of a PE wax, based on the total weight of the additive, wherein Bacillus licheniformis M0 is attached to the carrier.

Further, there is provided a method of preparing a polyolefin product additive, for preparing a polyolefin product additive in the form of a masterbatch, the method comprising the steps of:
S1: attaching, by atomization, a bacterial solution comprising Bacillus licheniformis M0 to a surface of a carrier;
S2: mixing the carrier with a polyolefin resin and a PE wax to obtain a mixture;
S3: processing the mixture into the polyolefin product additive using a screw extruder.

Preferably, the screw extruder in step S3 has an extrusion temperature of 170 to 210° C., more preferably 180 to 200° C.

Preferably, the amounts of the components in the mixture, in terms of weight percentage, range from 50% to 80% of the carrier, 16% to 46% of the polyolefin resin, and 4% of the PE wax.

Further, there is also provided a polyolefin product, comprising the polyolefin degradation microbial agent and a polyolefin material, wherein the polyolefin product additive is added in an amount of 10 to 50% by weight of the polyolefin product, and the polyolefin material is added in an amount of 50 to 90% by weight of the polyolefin product.

Preferably, the polyolefin product has a processing temperature of 180 to 280° C.

According to Bacillus licheniformis M0 of the present application, after the polyolefin product containing the strain is buried in compost or soil after use, or after the strain is sprayed in the discarded polyolefin product and then buried in compost or soil, the Bacillus licheniformis M0 can make the polyolefin product degraded in a natural environment and decomposed into simple compounds such as carbon dioxide, inorganic salts and water.

### BRIEF DESCRIPTION OF THE DRAWINGS

The following drawings are hereby incorporated as part of the present application for the understanding of the present application. The embodiments of the present application are illustrated and described in the drawings in order to explain the principles of the present application.

In the drawings:

FIG. 1 shows a photograph of colony morphology of Bacillus licheniformis M0;

FIG. 2 shows biodegradation percentage curves of experimental and reference materials.

### DETAILED DESCRIPTION

In the following description, numerous specific details are set forth in order to provide a more thorough understanding of the present application. However, it is obvious to those skilled in this art that the embodiments of the present application may be implemented without one or more of these details. Some technical features well-known in this art are not described in other examples in order to avoid confusion with the embodiments of the present application.

In order to thoroughly understand the present application, a detailed structure will be provided in the following description. Obviously, the implementation of the embodiments of the present application is not limited to the specific details familiar to those skilled in the art. The preferred embodiments of the present application are illustrated in detail below. However, the present application may further have other embodiments in addition to the detailed description.

It shall be noted that the terminology used herein is for the purpose of describing particular embodiments only and is not intended to limit the exemplary embodiments of the present application. As used herein, the singular forms are intended to include the plural forms as well, unless the context clearly indicates otherwise. It will be further understood that the terms “comprising” and/or “including,” when used in this specification, specify the presence of stated features, wholes, steps, operations, elements, and/or components, but do not preclude the presence or addition of one or more other features, wholes, steps, operations, elements, components, and/or combinations thereof.

Ordinals such as “first” and “second” quoted in this application are merely identifiers and do not carry any other meaning, such as a specific order. Moreover, for example, the term “first component” itself does not imply the presence of “second component”, and the term “second component” itself does not imply the presence of “first component.”

It should be noted that the terms “up”, “down”, “front”, “back”, “left”, “right”, “inside”, “outside” and similar expressions used herein are for illustrative purposes only and are not restrictive.

The exemplary embodiments of the present application will now be explained in further detail with reference to the accompanying drawings. However, these exemplary embodiments can be implemented in various forms and should not be construed as being limited to the embodiments set forth herein. It is to be understood that these embodiments are provided to make the disclosure thorough and complete and to make the concept of these exemplary embodiments completely delivered to those of ordinary skill in the art.

In one specific embodiment of the present disclosure, a polyolefin degrading bacterium, Bacillus licheniformis M0, is provided with the following information on deposit:
Name of the biological material: Bacillus licheniformis M0; Latin name: Bacillus licheniformis M0;
Depository Institution: Guangdong Microbial Culture Collection Center; Address: 5th Floor, Building 59, No. 100, Xianlie Middle Road, Guangzhou, Guangdong, China;
Date of Deposit: Feb. 8, 2023; accession number: GDMCC NO: 63160.
   A. Acquisition and Identification of Bacillus licheniformis M0

### 1. Acquisition of Native Strains

In the waste pile, different changes are observed on the surfaces of different plastic products, some of which apparently showed signs of microbial activity. The materials with the above obvious phenomenon are selected from different waste piles. The microbial communities on the surface are sampled and enriched, and the microbial enrichment liquid, also known as a bacterial liquid, is preliminarily obtained.

### 2. Screening of Efficient Bacterial Strains

Take the bacterial liquid, mix it well before use, and use a dilution coating plate method for isolation. Gradient dilution is performed with phosphate buffer (10⁻¹, 10⁻², 10⁻³, 10⁻⁴, 10⁻⁵, 10⁻⁶), then 100 μL of each gradient dilution is coated on the nutrient AGAR plate, and the two plates are determined in parallel and placed at 36° C.±1° C. for 24 h to 48 h incubation.

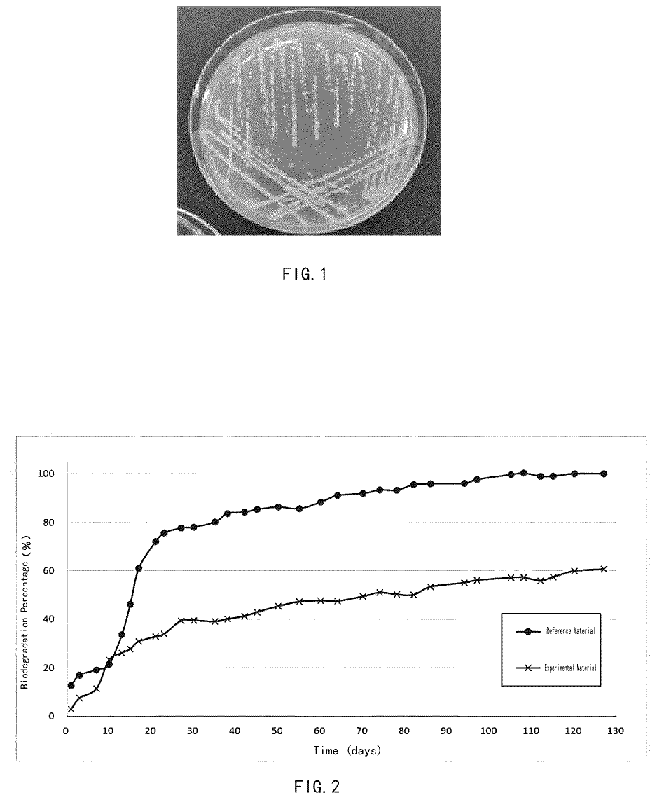

The colony growth morphology on the nutrient AGAR medium is observed. Single colonies of different morphology are selected from the nutrient AGAR plate, delineated on the nutrient AGAR plate, respectively, and cultured at 36° C.±1° C. for 24 h to 48 h for purification, and the purification is repeated for 3 times, finally obtaining Bacillus licheniformis M0. The colony morphology is shown in FIG. 1.

The ingredients of the nutrient AGAR medium comprise peptone (10.0 g), beef extract (3.0 g), sodium chloride (5.0 g), AGAR (15.0 g), and distilled water (1000 mL). Mix the above ingredients well, boil to dissolve, and adjust pH to 7.3±0.2. It is divided into test tubes or conical bottles and autoclaved at 121° C. for 15 min to obtain the nutrient AGAR medium.

The ingredients of the phosphate buffer solution include 34.0 g of potassium dihydrogen phosphate (KH₂PO₄) and 500 mL of distilled water. Specifically, 34.0 g of potassium dihydrogen phosphate is weighed and dissolved in 500 mL of distilled water, the pH is adjusted to 7.2 with about 175 mL of 1 mol/L sodium hydroxide solution, and then the storage solution is obtained by diluting it to 1,000 mL in distilled water and then storing it in a refrigerator. Take 1.25 mL of the storage solution, dilute it to 1000 mL with distilled water, dispense it into suitable containers, and autoclave it at 121° C. for 15 min to obtain the phosphate buffer solution.

3. Molecular Biological Identification of Bacillus licheniformis M0

The samples are preliminarily identified by DNA extraction of the strain samples, amplification and sequencing using bacterial 16S universal primers, and comparison of the sequencing results in the NCBI database.

Bacterial 16S rDNA sequences are highly conserved among different species, and the sequences are not the same among different species. Therefore, the bacterial species can then be determined after the sequences are amplified and sequenced by PCR and compared with the known sequences in GenBank, and the bacteria can be categorized into genera or species.

16S rDNA (16S rRNA is a subunit of ribosomal RNA, and 16S rDNA is the gene encoding the subunit) identification refers to the identification of bacterial species by using bacterial 16S rDNA sequence sequencing. 16S rDNA is the corresponding DNA sequence that encodes the 16S rRNA on bacterial chromosomes, which is present in all bacterial chromosome genes. rRNA refers to the transcription product of rDNA, which is an important component of the ribosome assembled by many small rRNA molecules, and 16S rRNA is one of the components. 16S rDNA is generally analyzed because DNA extraction is easy and relatively stable.

### I. DNA is Extracted Using a DNA Extraction Kit (General-Purpose), and the Specific Steps are as Follows:

(1) Put Spin Column in Collection Tube, add 250 μl Buffer BL, centrifuge at 12000 rpm/min for 1 min to activate silica gel membrane;
(2) Take 1 mL of bacterial solution into a 1.5 ml centrifuge tube, add 400 μl Buffer gP1, vortex and oscillate for 1 min, and water bathe at 65° C. for 10 to 30 min, during which time it can be reversed and mixed for full cracking;
(3) Add 150 μl Buffer gP2, vortex and oscillate for 1 min, and ice bathe for 5 min;
(4) Centrifuge at 12000 rpm/min for 5 min and transfer the supernatant to a new centrifuge tube;
(5) Add anhydrous ethanol in the same volume as the supernatant, immediately shake and mix thoroughly, transfer all the liquid into the Spin Column, centrifuge for 30 s at 12,000 rpm/min, and discard the waste liquid;
(6) Add 500 μl Buffer Pw (anhydrous ethanol is added before use) to the Spin Column, centrifuge at 12,000 rpm/min for 30 s, and discard the waste liquid;
(7) Add 500 μl Wash Buffer (anhydrous ethanol is added before use) to the Spin Column, centrifuge at 12000 rpm/min for 30 s, and discard the waste liquid;
(8) Repeat step 7;
(9) Put the Spin Column back into the Collection Tube, centrifuge at 12,000 rpm/min for 2 min, and open the lid to dry for 1 min;
(10) Remove the Spin Column, put it into a clean centrifuge tube, add 50 to 100 μl TE Buffer (65° C. preheated TE Buffer) in the center of the adsorbent membrane, place it at 20 to 25° C. for 2 min, and centrifuge at 12,000 rpm/min for 2 min.
   II. PCR amplification

The universal primers for strain identification are as below:

| | | | Amplified | |
|---|---|---|---|---|
| Category | Name | Sequence 5′->3′ | Sequence | PCR Length/bp |
| Bacteria | 27F | AGTTTGATCMTGGCTCA | 16S rDNA | about 1500 bp |
| | 1492R | GGTTACCTTGTTACGACTT | | |

The extracted DNA samples are diluted in appropriate amounts and used as PCR templates, which are amplified with DynaPro 1×TSE101 Gold mix, and each components of the amplification system are as follows:

| | |
|---|---|
| 1 × TSE101 Gold mix | 45 μl |
| 27F (10P) | 2 μl |
| 1492R (10P) | 2 μl |
| DNA template | 1 μl |

The above amplification system is amplified according to the following amplification procedure:

| Stage | Temperature | Time | | Number of cycles |
|---|---|---|---|---|
| Pre-denaturation | 98° C. | 2 | min | |
| Cycling phase | 98° C. | 10 | s | 35 cycles |
| | 56° C. | 10 | s | |
| | 72° C. | 10 | s/kb | |
| Extension phase | 72° C. | 5 | min | |

| | | |
|---|---|---|
| Preservation phase | 4° C. | — |

### III. Electrophoresis Detection

The amplified PCR products are subjected to agarose gel electrophoresis (2 μl of sample+6 μl of bromophenol blue) at 300 V for 12 minutes to obtain the identification gel graph. The prepared PCR products are sent to Beijing Prime Biotechnology Co., Ltd. in Guangzhou for one-generation sequencing, and the sequencing results are shown in SEQ ID NO:1:

(SEQ ID NO: 1)
TCAGACGAACGCTGGCGGCGTGCCTAATACATGCAAGTCGAGCGGACCGA
CGGGAGCTTGCTCCCTTAGGTCAGCGGCGGACGGGTGAGTAACACGTGGG
TAACCTGCCTGTAAGACTGGGATAACTCCGGGAAACCGGGGCTAATACCG
GATGCTTGATTGAACCGCATGGTTCAATCATAAAAGGTGGCTTTTAGCTA
CCACTTACAGATGGACCCGCGGCGCATTAGCTAGTTGGTGAGGTAACGGC
TCACCAAGGCGACGATGCGTAGCCGACCTGAGAGGGTGATCGGCCACACT
GGGACTGAGACACGGCCCAGACTCCTACGGGAGGCAGCAGTAGGGAATCT
TCCGCAATGGACGAAAGTCTGACGGAGCAACGCCGCGTGAGTGATGAAGG
TTTTCGGATCGTAAAACTCTGTTGTTAGGGAAGAACAAGTACCGTTCGAA
TAGGGCGGTACCTTGACGGTACCTAACCAGAAAGCCACGGCTAACTACGT
GCCAGCAGCCGCGGTAATACGTAGGTGGCAAGCGTTGTCCGGAATTATTG
GGCGTAAAGCGCGCGCAGGCGGTTTCTTAAGTCTGATGTGAAAGCCCCCG
GCTCAACCGGGGAGGGTCATTGGAAACTGGGGAACTTGAGTGCAGAAGAG
GAGAGTGGAATTCCACGTGTAGCGGTGAAATGCGTAGAGATGTGGAGGAA
CACCAGTGGCGAAGGCGACTCTCTGGTCTGTAACTGACGCTGAGGCGCGA
AAGCGTGGGGAGCGAACAGGATTAGATACCCTGGTAGTCCACGCCGTAAA
CGATGAGTGCTAAGTGTTAGAGGGTTTCCGCCCTTTAGTGCTGCAGCAAA
CGCATTAAGCACTCCGCCTGGGGAGTACGGTCGCAAGACTGAAACTCAAA
GGAATTGACGGGGGCCCGCACAAGCGGTGGAGCATGTGGTTTAATTCGAA
GCAACGCGAAGAACCTTACCAGGTCTTGACATCCTCTGACAACCCTAGAG
ATAGGGCTTCCCCTTCGGGGGCAGAGTGACAGGTGGTGCATGGTTGTCGT
CAGCTCGTGTCGTGAGATGTTGGGTTAAGTCCCGCAACGAGCGCAACCCT
TGATCTTAGTTGCCAGCATTCAGTTGGGCACTCTAAGGTGACTGCCGGTG
ACAAACCGGAGGAAGGTGGGGATGACGTCAAATCATCATGCCCCTTATGA
CCTGGGCTACACACGTGCTACAATGGGCAGAACAAAGGGCAGCGAAGCCG
CGAGGCTAAGCCAATCCCACAAATCTGTTCTCAGTTCGGATCGCAGTCTG
CAACTCGACTGCGTGAAGCTGGAATCGCTAGTAATCGCGGATCAGCATGC
CGCGGTGAATACGTTCCCGGGCCTTGTACACACCGCCCGTCACACCACGA
GAGTTTGTAACACCCGAAGTCGGTGAGGTAACCTTTTGGAGCCAGCCGCC
GAAGGTGGGACAGATGATTGGGGTGAAGTCGTAACAGGGTAGCCGTA.

Comparison of the above sequences with known sequences in GenBank shows that the 16S rDNA gene sequences are 99% similar to those of several species of Bacillus licheniformis, and that the strain belongs to the genus Bacillus licheniformis.

In order to maintain the activity of strains, liquid ammonia ultra-low temperature cryopreservation of the strains is carried out by using methods known in the art (e.g., with reference to Compendium of Technical Regulations for the Collection, Arrangement and Preservation of Microbial Strain Resources (Publication date: May 2011, published by the China Agricultural Science and Technology Press)) and employing 10 to 20% glycerol as a protectant, and the strains are kept in a frozen preservation state at all times until they are used. Other common microbial strain protectants such as dimethyl sulfoxide, dextrin, serum proteins, polyethylene azetidine, tween 80, etc., can also be used in the cryopreservation of the strains, as long as they can maintain the activity of the strains at low temperatures.

### B. Preparation and Degradation Detection of Polyolefin Products

Bacillus licheniformis M0 can be used to prepare a polyolefin degradation microbial agent, which can be prepared in a variety of dosage forms, such as liquid, emulsion, suspension, powder, granule, wettable powder or water dispersible granule.

The polyolefin degradation microbial agent can be used to degrade common polyolefin products in the environment, and can also be added to polyolefin products as a polyolefin product additive in the production process to prepare degradable polyolefin products.

Bacillus licheniformis M0 is added in the polyolefin product in an amount of more than or equal to 10 cfu/g relative to the total weight of the polyolefin product.

Bacillus licheniformis M0 is added in the polyolefin product in an amount of 10³ to 5×10⁴ cfu/g relative to the total weight of the polyolefin product. When the addition amount is lower than this range, it will lead to insufficient microbial content in the final product, and although the polyolefin can still degrade, the efficiency will be significantly reduced, so it is not preferred. When the addition amount is higher than this range, the cost may be too high and uneconomical.

### 1. Preparation of Polyolefin Product Additives in the Form of Masterbatches

S1: A bacterial solution including Bacillus licheniformis M0 is attached to the surface of a carrier by atomization.

S2: The carrier, a polyolefin resin and a PE wax are mixed to obtain a mixture, and the temperature is maintained between 6° and 70° C. during the mixing process, wherein the amount of each component in the mixture, by weight percentages, is: 50 to 80% of the carrier, 16 to 46% of the polyolefin resin, and 4% of the PE wax.

S3: The mixture is made into a polyolefin product additive, i.e., a masterbatch, using a screw extruder, and the screw extruder has an extrusion temperature of 170 to 210° C., preferably 180 to 200° C.

S4: After extrusion, the polyolefin product additive is cooled with water by pulling strips, and then the polyolefin product additive is cut into granules by a high-speed granulator to obtain the polyolefin product additive in the form of masterbatch.

During the process, the carrier, the polyolefin resin and the PE wax may be of known or commercially available materials.

The carrier may be a solid carrier. The solid carrier can be further categorized into mineral materials and plant materials. The mineral material may be at least one of clay, talc, kaolin, montmorillonite, white carbon, zeolite, silica, calcium carbonate and diatomaceous earth. The plant material may be at least one of bamboo flour, wood flour, corn flour, soybean flour, and starch. In one embodiment, the carrier is calcium carbonate.

The polyolefin resin may be Saudi LLDPE or other polyolefin resin.

Furthermore, it is preferable to add substances such as sugars and chitin to the above polyolefin product additive to facilitate metabolic activity after subsequent recovery of the strain.

The amount of Bacillus licheniformis M0 added to the prepared polyolefin product additive is more than or equal to 100 cfu/g relative to the total weight of the polyolefin product additive.

Preferably, the amount of Bacillus licheniformis M0 added to the prepared polyolefin product additive is 10⁴ to 10⁵ cfu/g relative to the total weight of the polyolefin product additive.

### 2. Preparation of Polyolefin Products

After the polyolefin product additive in the form of masterbatch is blended with the polyolefin material, the polyolefin product is made by conventional preparation methods such as injection molding, blister molding, blow molding, molding, extrusion, and so on, and the product is processed at a temperature of 180 to 280° C. In the polyolefin product by weight percentages, the addition amount of the polyolefin product additive in the form of masterbatches is 10 to 50% of the total weight of the polyolefin product, and the addition amount of the polyolefin material is 50 to 90% of the total weight of the polyolefin product.

The polyolefin material may be Saudi LLDPE or other polyolefin materials.

### 3. Degradation Detection

This embodiment is to determine the biodegradation percentage of experimental materials in a composting environment. The experimental material is introduced into a composting vessel and subjected to intense aerobic composting at a defined temperature, oxygen concentration and humidity. During aerobic biodegradation of the experimental material, carbon dioxide, water, mineralized inorganic salts and new biomass are all the final biodegradation products. The carbon dioxide produced by the experimental and blank containers is monitored continuously and measured periodically during the experiment, and the carbon dioxide produced is calculated cumulatively. The ratio of the actual amount of carbon dioxide produced by the experimental material during the experiment to the theoretical amount of carbon dioxide that can be produced by that material is the biodegradation percentage. The theoretical amount of CO₂ released can be calculated from the actual measured total organic carbon (TOC) content.

The reference material is a degradable material that is subjected to simultaneous degradation tests under the same conditions to verify the validity of the test.

Specific test supply information is provided below:

| Test material | Degradable polyolefin products |
|---|---|
| Reference | Thin layer chromatography cellulose, with |
| material | a particle size less than 20 μm |
| Compost | Compost produced from farmyard manure, |
| source | 3 months old |
| Carbon dioxide | Carbon dioxide emitted from the vessel |
| test method | is absorbed using a 20 g/L sodium hydroxide |
| | solution, and then a total organic carbon |
| | analyzer is used to determine inorganic carbon |
| | and finally the amount of carbon dioxide. |
| Compost container | Glass material, capacity 3 L |
| Constant | 58 ± 2° C. |
| temperature oven | |

In the process, a stock solution of Bacillus licheniformis M0 with a concentration of 108 cfu/ml is configured into a dilution solution with a concentration of 106 cfu/ml, and 100 ml is configured. 100 ml of the dilution solution is attached to the surface of 800 g of the carrier by atomization. The 800 g of carrier, 160 g of Saudi LLDPE and 40 g of PE wax are mixed through a high-speed co-mixer at 800 rpm between 6° and 70° C. for 5 minutes to obtain a mixture. The mixture is extruded through a twin-screw extruder at 170 to 210° C., preferably 180 to 200° C., and 1 kg of polyolefin product additive is prepared by pelletizing, with the amount of Bacillus licheniformis M0 added at 10⁵ cfu/g relative to the total weight of the polyolefin product additive. 300 g of polyolefin product additives and 700 g of Saudi LLDPE (i.e., 30% polyolefin product additives and 70% polyolefin materials by weight percentage) are blended, polyolefin products are prepared as experimental materials through a film blowing process, and the amount of Bacillus licheniformis M0 added is 3×10⁴ cfu/g relative to the total weight of the polyolefin products.

The basic properties of the experimental and reference materials are as follows:

| | Experimental | Reference |
|---|---|---|
| | Material | Material |
| Shape | Granular | Powder |
| Size | Less than 2 cm × 2 cm | — |
| Sample size (g) | 50 | 50 |
| Total dry solids (%) | 99.8 | 97.6 |
| Humidity (%) | 0.2 | 2.4 |
| Total Organic Carbon | 46.82 | 44.40 |
| (TOC, %) | | |
| Theoretical carbon | 85.66 | 79.45 |
| dioxide release (ThCO₂, g) | | |

Prepare at least the following number of compost containers:
a) 3 containers for experimental material;
b) 3 containers for reference material; and
c) 3 blank containers.

The experimental and reference materials are introduced into the corresponding compost containers, respectively, and the blank containers contain only compost, with test conditions of 58±2° C. and an aeration flow rate of 100 to 150 ml/min.

During the experiment, the cumulative release of carbon dioxide is continuously detected, and the humidity of the compost mixture is continuously maintained at 50%, while the changes in the test samples during composting are continuously monitored.

The percentage of biodegradation is calculated based on the cumulative amount of carbon dioxide released using the following formula:

$${D}_{t} = [ {( {CO}_{2} )}_{T} - {( {CO}_{2} )}_{B} ] / {ThCO}_{2} \times 100$$

Wherein Dt represents the biodegradation percentage (%) of the experimental or reference material;
(CO2)_{T} represents the cumulative amount of carbon dioxide released from each compost container containing the experimental or reference material, in grams per container (g/container);
(CO2)_{B} represents the average of the cumulative amount of carbon dioxide emitted from blank containers, in grams per container (g/container);
ThCO₂ represents the theoretical release of carbon dioxide produced by the experimental or reference material, in grams per container (g/container).

The experimental results are shown in FIG. 2 and the table as below:

| Days | (CO₂)_{B1} | (CO₂)_{B2} | (CO₂)_{B3} | (CO₂)_{B} | (CO₂)_{T1} | (CO₂)_{T2} | (CO₂)_{T3} | Dₜ₁ | Dₜ₂ | Dₜ₃ | Dₜ |
|---|---|---|---|---|---|---|---|---|---|---|---|
| 10 | 24.34 | 24.05 | 26.76 | 25.05 | 40.03 | 45.34 | 45.04 | 17.49 | 23.69 | 23.34 | 21.50 |
| 45 | 38.64 | 40.86 | 46.08 | 41.86 | 76.13 | 83.09 | 76.59 | 40.00 | 48.13 | 40.54 | 42.89 |
| 127 | 52.49 | 50.21 | 62.40 | 55.03 | 106.11 | 106.63 | 108.20 | 59.63 | 60.23 | 62.06 | 60.64 |

$$Wherein , {( {CO}_{2} )}_{B} = [ {( {CO}_{2} )}_{B 1} + {( {CO}_{2} )}_{B 2} + {( {CO}_{2} )}_{B 3} ] / 3$$

$${D}_{ti} = [ {( {CO}_{2} )}_{Ti} - {( {CO}_{2} )}_{B} ] / {ThCO}_{2} \times 100$$

$${D}_{t} = [ {D}_{t1} + {D}_{t2} + {D}_{t3} ] / 3$$

It can be seen from the table that the biodegradation percentages of polyolefin products as experimental materials are 21.50%, 42.89% and 60.64% at 10, 45 and 127 days, respectively. From FIG. 2, it can be seen that the biodegradation percentage of the reference material after 45 days is more than 70%, proving that the test is effective.

Obviously, the polyolefin products prepared with the polyolefin product additives containing Bacillus licheniformis M0 can be degraded to simple compounds such as carbon dioxide, inorganic salts and water in a compost or soil environment. In contrast, the polyolefin products without the strain are non-degradable in the same environment.

### C. Physical Property Testing of Polyolefin Products

The above polyolefin products prepared by adding the polyolefin product additives, 50 mm in length, 12 mm in width, and 0.035 mm in thickness, are tested for mechanical properties, and the physical properties are shown in the table as below:

| | Tensile | Elongation | Yield | Yield | Puncture | |
|---|---|---|---|---|---|---|
| Specimen | strength | at break | stress | elonga- | force | Judg- |
| No. | (MPa) | (%) | (MPa) | tion (%) | (N) | ment |
| 1 | 26.980 | 360.1 | 20.48 | 348.1 | 2.24 | PASS |
| 2 | 27.277 | 356.7 | 27.26 | 348.0 | 2.22 | PASS |
| 3 | 28.390 | 355.6 | 28.38 | 338.1 | 2.27 | PASS |
| Mean | 27.549 | 357.5 | 25.37 | 344.7 | 2.24 | PASS |
| Variance | 0.743 | 2.3 | 4.27 | 5.7 | 0.03 | PASS |

The biodegradable shopping bags produced using the processes and techniques described in the present disclosure achieve degradation properties while still maintaining the usability properties of conventional plastics.

Referring to a series of standard tests of GB/T 21661-2020 Plastic Shopping Bags, the shopping bags prepared by the present disclosure fully satisfy the requirements of the mechanical properties of ordinary plastic shopping bags, and will not sacrifice the usability in order to meet the degradation conditions.

With the addition of the degrading strain Bacillus licheniformis M0 to the polyolefin product, the polyolefin product has the same shelf life as the general-purpose polyolefin product in the environment of daily use and still has good physical properties. The polyolefin product not only retains the durability of polyolefin, but also is degradable, which makes it cost-effective.

The order of the steps of the method of the embodiments of the present disclosure may be adjusted, combined, or deleted according to practical needs. The units of the terminal of the embodiments of the present disclosure may be integrated, further divided or abridged according to actual needs.

The processes described in all of the preferred embodiments described above are only exemplary. Various processing operations may be performed in an order different from the order of the above-described processes unless an unfavorable effect occurs. The order of the steps of the above-described processes may also be added, combined or deleted as desired.

In addition, the commands, command numbers, and data items described in all of the above-described preferred embodiments are only examples, and thus these commands, command numbers, and data items may be set in any manner as long as the same function is realized. The units of the terminals of each preferred embodiment may also be integrated, further divided or deleted according to actual needs.

Unless otherwise defined, the technical and scientific terms used herein have the same meanings as commonly understood by those skilled in the technical field of the present application. The terms used herein are only for describing specific implementation purposes, and are not intended to limit the present application. A feature described in one embodiment herein can be applied to another embodiment alone or in combination with other features, unless the feature is not applicable in the other embodiment or otherwise stated.

The present application has been described through the above-mentioned embodiments, but it should be understood that the above-mentioned embodiments are only for the purpose of illustration and description, and are not intended to limit the present application to the described embodiments. More variations and modifications can be made according to the teachings of the present application, and these variations and modifications fall within the protection scope claimed by the present application.

1. Bacillus licheniformis (Bacillus licheniformis) M0, deposited in Guangdong Microbial Culture Collection Center on Feb. 8, 2023, with the accession number being GDMCC NO: 63160.

2. Use of Bacillus licheniformis M0 according to claim 1, in degrading polyolefin products.

3. The use according to claim 2, wherein Bacillus licheniformis M0 is added in the polyolefin product in an amount of more than or equal to 10 cfu/g relative to the total weight of the polyolefin product.

4. The use according to claim 3, wherein Bacillus licheniformis M0 is added in the polyolefin product in an amount of 103 to 5×104 cfu/g relative to the total weight of the polyolefin product.

5. A polyolefin degradation microbial agent, comprising Bacillus licheniformis M0 of claim 1.

6. The polyolefin degradation microbial agent according to claim 5, wherein the polyolefin degradation microbial agent is a polyolefin product additive in the form of a masterbatch.

7. The polyolefin degradation microbial agent according to claim 6, wherein the polyolefin product additive in the form of the masterbatch comprises 50 to 80% of a carrier, 16 to 46% of a polyolefin resin, and 4% of a PE wax, based on the total weight of the additive, wherein Bacillus licheniformis M0 is attached to the carrier.

8. The polyolefin degradation microbial agent according to claim 7, wherein the carrier is at least one selected from clay, talc, kaolin, montmorillonite, white carbon, zeolite, silica, calcium carbonate or diatomaceous earth.

9. The polyolefin degradation microbial agent according to claim 5, comprising a protective agent for ultra-low temperature cryopreservation of liquid nitrogen.

10. A method of preparing a polyolefin product additive in the form of a masterbatch, comprising the steps of:
S1: attaching, by atomization, a bacterial solution comprising Bacillus licheniformis M0 of claim 1 to a surface of a carrier;
S2: mixing the carrier with a polyolefin resin and a PE wax to obtain a mixture; and
S3: processing the mixture into the polyolefin product additive using a screw extruder.

11. The method according to claim 10, wherein the screw extruder in step S3 has an extrusion temperature of 170 to 210° C.

12. The method according to claim 10, wherein the amounts of the components in the mixture, in terms of weight percentage, range from 50 to 80% of the carrier, 16 to 46% of the polyolefin resin, and 4% of the PE wax.

13. The method according to claim 10, wherein the bacterial solution comprises a protective agent for ultra-low temperature cryopreservation of liquid nitrogen.

14. A polyolefin product, comprising the polyolefin degradation microbial agent of claim 6 and a polyolefin material, wherein the polyolefin product additive is added in an amount of 10 to 50% by weight of the total weight of the polyolefin product and the polyolefin material is added in an amount of 50 to 90% by weight of the total weight of the polyolefin product.

15. The polyolefin product according to claim 14, wherein the polyolefin product has a processing temperature of 180 to 280° C.


SEQUENCE LISTING (1 sequence)

SEQ ID NO: 1 - DNA, 1497 bp, Bacillus licheniformis
  source 1..1497: mol_type = genomic DNA
tcagacgaacgctggcggcgtgcctaatacatgcaagtcgagcggaccgacgggagcttgctcccttaggtcagcggcggacgggtgagtaacacgtgggtaacctgcctgtaagactgggataactccgggaaaccggggctaataccggatgcttgattgaaccgcatggttcaatcataaaaggtggcttttagctaccacttacagatggacccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaaactctgttgttagggaagaacaagtaccgttcgaatagggcggtaccttgacggtacctaaccagaaagccacggctaactacgtgccagcagccgcggtaatacgtaggtggcaagcgttgtccggaattattgggcgtaaagcgcgcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggcgcgaaagcgtggggagcgaacaggattagataccctggtagtccacgccgtaaacgatgagtgctaagtgttagagggtttccgccctttagtgctgcagcaaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaaccctagagatagggcttccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatgggcagaacaaagggcagcgaagccgcgaggctaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaaccttttggagccagccgccgaaggtgggacagatgattggggtgaagtcgtaacagggtagccgta